(19) United States
(12) Patent Application Publication
(10) Pub. No.: US 2015/0164428 A1
(43) Pub. Date: Jun. 18, 2015

(54) Method for Multi-Scale Quality Assessment for Variability Analysis

(71) Applicants: OTTAWA HOSPITAL RESEARCH INSTITUTE, Ottawa (CA); THERAPEUTIC MONITORING SYSTEMS (TMS) INC., Ottawa (CA)

(72) Inventors: Daphne-Isabelle TOWNSEND, Embrun (CA); Christophe L. HERRY, Fournier (CA); Andrea BRAVI, Ottawa (CA); Geoffrey C. GREEN, Ottawa (CA); Andrew J.E. SEELY, Ottawa (CA)

(21) Appl. No.: 14/632,679

(22) Filed: Feb. 26, 2015

Related U.S. Application Data
(63) Continuation of application No. PCT/CA2013/050681, filed on Sep. 5, 2013.
(60) Provisional application No. 61/697,075, filed on Sep. 5, 2012.

(51) Int. Cl. A61B 5/00; G01R 13/00; A61B 5/0452; G01R 29/26

(52) U.S. Cl. CPC A61B 5/7221; G01R 29/26; G01R 13/00; A61B 5/0452; A61B 5/7282

(57) ABSTRACT

A system and method are provided for assessing quality for a variability analysis. The method comprises: obtaining at least one waveform corresponding to a corresponding physiological measurement; determining at least one measure of waveform quality of the at least one waveform; extracting from a waveform, at least one event time series; determining a measure of event time series quality of the at least one event time series; determining at least one measure of stationarity of the at least one event time series; computing a quality measure using the at least one measure of waveform quality and the at least one measure of stationarity; and displaying the quality measure.

### CROSS-REFERENCE TO RELATED APPLICATIONS

This application is a continuation of PCT Application No. PCT/CA2013/050681 filed on Sep. 5, 2013, which claims priority to U.S. Provisional Application No. 61/697,075 filed on Sep. 5, 2012, both incorporated herein by reference.

### TECHNICAL FIELD

The following relates to performing quality assessments for variability analyses.

### DESCRIPTION OF THE RELATED ART

Physiological waveforms are now recorded at the bedside for the purposes of storage and analysis. This valuable data can be used for retrospective review, and can also be processed and used by display and decision support algorithms. The challenge is shifting from data availability towards data quality: i.e., removing noise and artifacts and finding relevant information within the surplus of available data. Computerized data collection systems now allow for the analysis of vast quantities of data. To ensure reliability and fidelity of results provided by the intersection of computer science and health care, a quality assessment should be in place to ensure the waveforms are appropriately processed (G. Takla, J. H. Petre, D. J. Doyle, M. Horibe and B. Gopakumaran, “The problem of artifacts in patient monitor data during surgery: a clinical and methodological review,” Anesth. Analg., vol. 103, pp. 1196-1204, November 2006).

This process, which combines the flexibility of a visual inspection with objective physiological measurements identifies and removes signal features which may compromise the quality of downstream processing.

The science of variability measurement is gaining recognition for its importance as an indicator of illness severity and for its possible diagnostic use. The importance of examining waveforms and events (i.e. time series extracted from those waveforms) prior to variability measurement has been widely observed (see: i) Q. Li, R. G. Mark and G. D. Clifford, “Robust heart rate estimation from multiple asynchronous noisy sources using signal quality indices and a Kalman filter,” Physiol. Meas., vol. 29, pp. 15-32, January 2008; ii) T. C. Smith, A. Green and P. Hutton, “Recognition of cardiogenic artifact in pediatric capnograms,” J. Clin. Monit., vol. 10, pp. 270-275, July 1994; and iii) V. Papaioannou, C. Dragoumanis and I. Pneumatikos, “Biosignal analysis techniques for weaning outcome assessment,” J. Crit. Care, vol. 25, pp. 39-46, March 2010) and presenting one or more variability measurements to clinicians may provide a better understanding of the complexity of event patterns (see: i) I. Jabloński, K. Subzda and J. Mroczka, “Software Tool for Assessment of Complexity and Variability in Physiological Signals of Respiration,” International Journal of Measurement Technologies and Instrumentation Engineering, vol. 28, pp. 28, 2011; and ii) M. F. El-Khatib, “A diagnostic software tool for determination of complexity in respiratory pattern parameters,” Comput. Biol. Med., vol. 37, pp. 1522, 2007).

In 1996, the Task Force of the European Society of Cardiology the North American Society of Pacing published recommendations and highlighted the clinical relevance and applicability of HRV (Electrophysiology, Task Force of the European Society of Cardiology the North American Society of Pacing, “Heart Rate Variability Standards of Measurement, Physiological Interpretation, and Clinical Use,” Circulation, vol. 93, pp. 1043-1065, March 1996). Ten years later, an IEEE review drew attention to the significance of measuring HRV for the monitoring of sepsis, exercise, post myocardial infarction patients and sepsis in adults, as fetal distress and apnea in neonates (S. Cerutti, A. L. Goldberger and Y. Yamamoto, “Recent Advances in Heart Rate Variability Signal Processing and Interpretation,” IEEE Transactions on Biomedical Engineering, vol. 53, pp. 1, January 2006). Ever since, measurements of complexity have expanded to include numerous measures in the statistical, geometric, energetic, informational and invariant domains (A. Bravi, A. Longtin and A. J. Seely, “Review and classification of variability analysis techniques with clinical applications,” Biomed. Eng. Online, vol. 10, pp. 90, Oct. 10. 2011).

### SUMMARY

It has been recognized that the clinician viewing variability results needs to know the quality of the variability measurements, and the quality of the waveform and event time series that was used to calculate that variability. It has also been found that there is a need to measure the quality of the data presented to the clinician in an automated and reliable fashion since relying on a visual inspection of waveform is typically insufficient.

To address the above, the following provides a modular framework to assess the quality of an input, event time series and variability measures, for the purpose of variability analysis. The method relates generally to medical monitoring and specifically to a quality assessment for the purpose of variability monitoring. The analysis comprises of a comprehensive quality assessment including assessing the quality at the waveform, event, stationarity and variability measurement level. A use of the quality measurements in the display variability measurement over time in the vicinity of a specific clinical event is also presented.

In one aspect, there is provided a method of assessing quality for a variability analysis, the method comprising: obtaining at least one waveform corresponding to a corresponding physiological measurement; determining at least one measure of waveform quality of the at least one waveform; extracting from a waveform, at least one event time series; determining a measure of event time series quality of the at least one event time series; determining at least one measure of stationarity of the at least one event time series; computing a quality measure using the at least one measure of waveform quality and the at least one measure of stationarity; and displaying the quality measure.

In another aspect, the method further comprises at least one of: performing a variability analysis on the at least one event time series at least one waveform, and displaying variability data, wherein the quality measure is displayed with variability data; computing a quality index using the at least one measure of waveform quality, wherein the quality index is computed using at least one of a threshold applied to a range for the quality measure and a mathematical model; wherein determining the at least one measure of waveform quality further comprises performing physiological filtering to remove at least one event from the at least one event time series; wherein determining the at least one measure of waveform quality further comprises removing at least one segment of a waveform signal; wherein determining the at least one measure of stationarity comprises removing at least one segment of the time series; wherein determining the quality measure further comprises performing event filtering on events detected from the at least one waveform; wherein determining the waveform quality comprises analyzing the at least one waveform for at least one of disconnections, saturations in the signal, and wandering baselines; and wherein the at least one waveform is displayed with the quality measure.

In yet another aspect, there is provided a computer readable storage medium comprising computer executable instructions for performing the method.

In yet another aspect, there is provided a system comprising a processor and memory, the memory comprising computer executable instructions for performing the method.

### BRIEF DESCRIPTION OF THE DRAWINGS

Embodiments will now be described by way of example only with reference to the appended drawings wherein:

FIG. 1 is a block diagram illustrating the incorporation of quality measurements into a variability analysis;

FIG. 2 is a flow diagram illustrating a process for quality assessment;

FIG. 3 is a representative diagram of quality framework for variability quality assessment;

FIG. 4 is a block diagram of a hospital site including a variability analysis server having a quality module;

FIG. 5 is a block diagram of a clinic site including a variability analysis server having a quality module;

FIG. 6 is a block diagram of a mobile site including a variability analysis server having a quality module;

FIG. 7 is a screen shot of a display of a single breath for expert annotation;

FIG. 8 illustrates a minute-long capnogram signal with non-physiological and high quality events identified;

FIG. 9 provides a histogram and cumulative distribution of the stationarity assessment for 5 minute windows;

FIG. 10 provides a screen shot of a display of a per window quality report;

FIG. 11 provides an example of possible quality report for end tidal CO2 signal using 5 minute windows with 0% overlap;

FIG. 12 provides a histogram of composite quality measure leading to thresholds to create quality index;

FIG. 13 provides an example of possible quality report for ECG signal using 5 minute windows with 25% overlap; and

FIG. 14 is a flow chart illustrating an example of a set of computer executable instructions that may be executed in performing a quality assessment on variability data.

### DETAILED DESCRIPTION

It will be appreciated that for simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures and components have not been described in detail so as not to obscure the examples described herein. Also, the description is not to be considered as limiting the scope of the examples described herein.

It will be appreciated that the examples and corresponding diagrams used herein are for illustrative purposes only. Different configurations and terminology can be used without departing from the principles expressed herein. For instance, components and modules can be added, deleted, modified, or arranged with differing connections without departing from these principles.

### Section I

### Overview

Physiological waveforms are now harvested at the bedside and manipulated to provide informational and decisional data points for clinicians and caregivers. For example, the study of heart rate variability (HRV) which is derived from the electrocardiogram (ECG) has benefited from nearly two decades of research and its applications in clinical practice are wide ranging. HRV is widely studied and used as a marker of illness severity.

Variability analysis measures the complexity of a time series of event occurrences, such as heart beats or breaths. It has been recognized that assessing the quality of the events, and the underlying waveform from which the events are derived is important to validate the subsequent interpretation of the variability measurements. The quality of the variability measurements themselves is also important in providing confidence in the reported values.

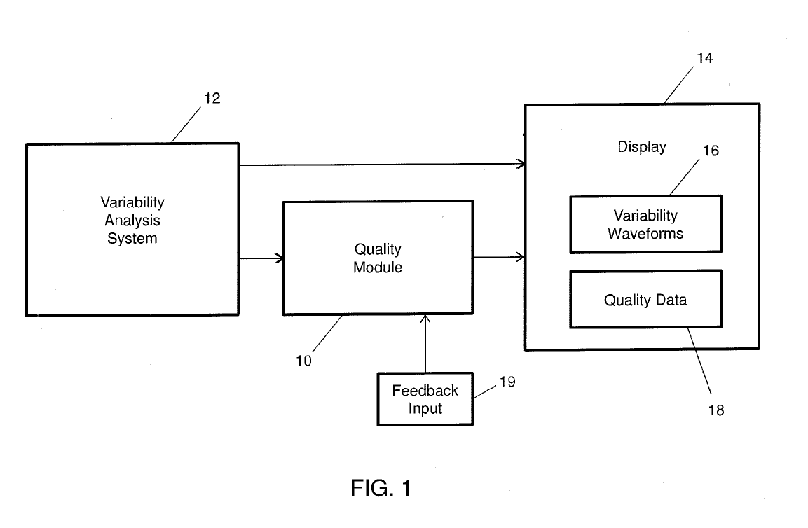

FIG. 1 illustrates a variability monitoring/analysis environment in which a quality module 10 is incorporated into or otherwise coupled to or integrated with a variability analysis system 12. Through this integration with the variability analysis system 12, the quality module 10 can generate quality data 18 (e.g., reports, measures, etc.) that can be displayed along with variability waveforms 16 on a display 14 of a computing system. The quality module 10 may also be configured, as shown in FIG. 1, to receive feedback input 19, such as user assessments of the quality of a particular variability waveform, interval or individual measurement, which can enhance the quality data 18.

It can be appreciated that the components in FIG. 1 are shown in isolation for illustrative purposes only and such components may be configured in different arrangements. For example, the components shown in FIG. 1 may be integrated into a single computing device or may operate within a distributed or otherwise networked system.

The present quality assessment therefore includes a modular framework for the analysis of a generic physiological waveform, and may also include event and stationarity assessments to prepare a high quality event time series for a variability analysis, and to measure the quality of the reported variability measures. The overall quality of the window can be reported as an index which summarizes the quality of the data at each step in processing. The framework described herein is also applied to the capnogram which is one embodiment of the method.

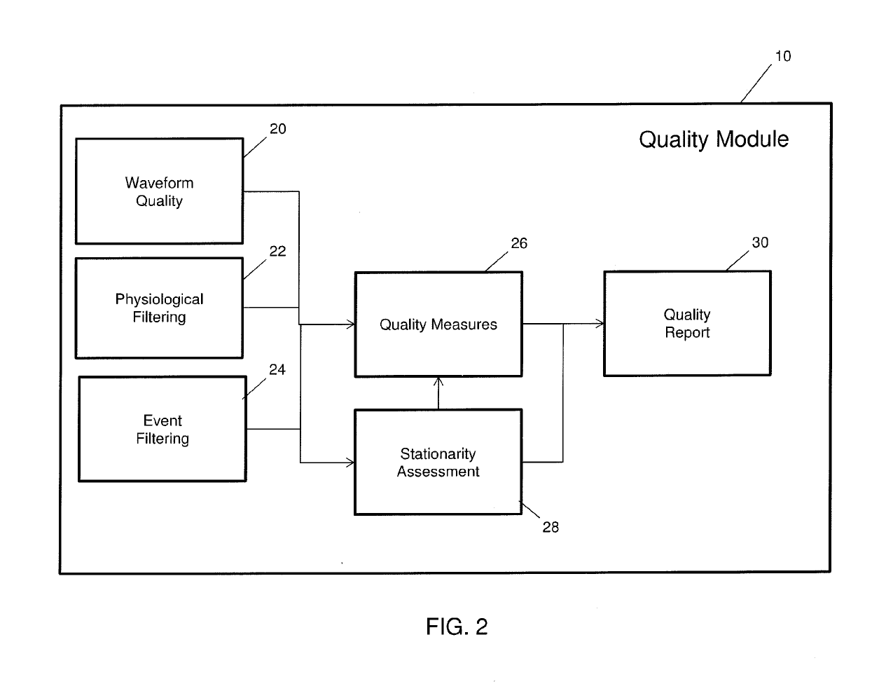

FIG. 2 illustrates further detail of a configuration for the quality module 10. In the example shown in FIG. 2, the quality module 10 performs a waveform quality stage 20, a physiological filtering stage 22, and an event filtering stage 24, all of which feed into a stationarity assessment 28 and a quality measures stage 26. The quality measures and stationarity assessments are then fed into a quality report stage 30, which reports on the quality of the variability waveforms being displayed, to enhance the data conveyed by such waveforms.

The following provides a quality assessment, addressing specific concerns for variability analysis. One embodiment uses the end tidal CO2 signal as an input waveform presented in section III.

The quality stages shown in FIG. 2 may be specifically designed for the purpose of variability analysis conducted over time as is its application to the capnogram signal. These quality stages can be used to ensure events used for variability calculations are of high quality and exhibit stationary behavior over a suitable period of time. The quality stages can be used to estimate the quality of the signal (i.e. level of noise and artifacts such as disconnections, saturation, baseline wandering, motion artifacts, etc.), and to exclude from the analysis segments of the signal that are of poor quality (thereby not enabling a proper computation of the event time series). A particular concern of stationarity which is assessed in the stationarity assessment stage 28, and which is of great importance for variability, is also addressed with these quality stages. It may be noted that in its simplest form, stationarity is the property of having stable statistical moments. It is recognized that a requirement for many popular techniques of time series analysis, including complexity of the variability measures. Without stationarity, interpreting the measures with confidence may be challenging (see R. Manuca and R. Savit, “Stationarity and nonstationarity in time series analysis,” Physica D, vol. 99, pp. 134-161, 12/15. 1996).

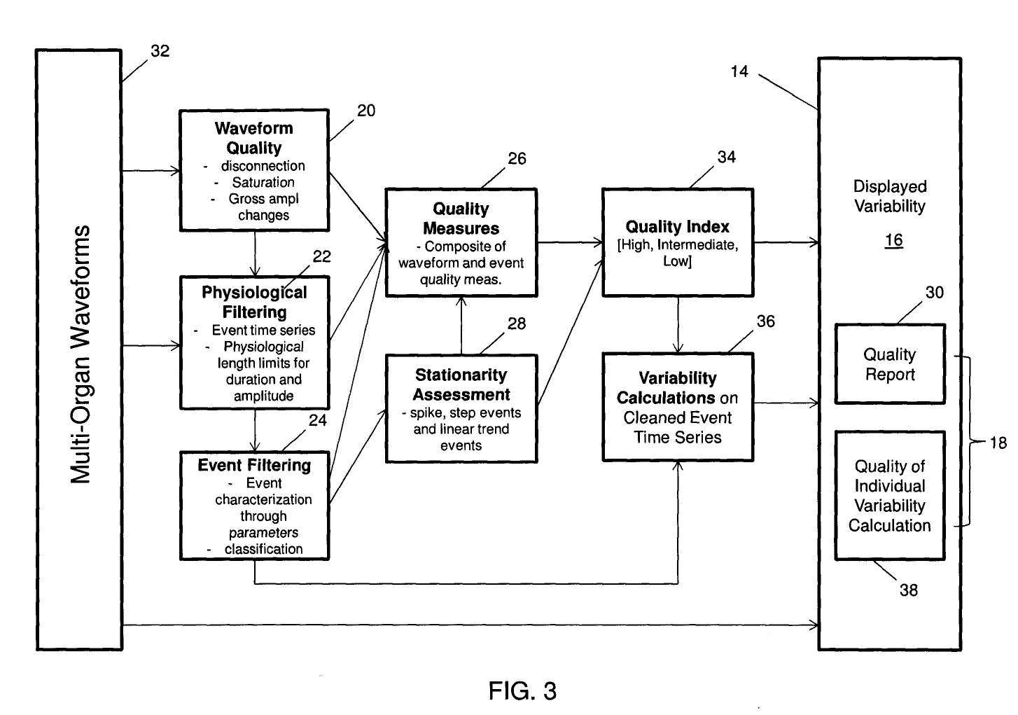

In a variability analysis, variability is calculated over time on the high quality event time series, usually on a plurality of windows, which may overlap. A quality assessment for variability may also be provided for variability measures calculated in time periods surrounding a clinical event. Therefore combining the waveform and event quality measures over a window provides a more complete quality assessment. The diagram of the assessment is presented in FIG. 2, and a detailed representative diagram for the quality assessment as it is integrated with the variability analysis, is presented in FIG. 3. In FIG. 2, as noted above, the processing stages include assessing the quality of the input waveform to identify segments which are suitable for segmentation into events. Following the segmentation into events, these events are stratified into three categories: non-physiological event, physiological events, and high quality events. Multiple methods for this stratification are described in section II.

As illustrated in FIG. 3, using time intervals (windows), the high quality event time series is assessed for stationarity in the stationarity assessment stage 28. The stationarity assessment stage 28 has been found to be important for variability measurements. Data obtained from the initial processing stages (waveform quality stage 20, physiological filtering stage 22, event filtering stage 24, and stationary assessment stage 28) are used to create quality measures in the quality measures stage 26. The quality measures are related to both a window (or interval) and to the individual variability measurements within that window. The quality measures may be implemented using a machine learning model (e.g. using neural networks, etc.) to optimally combine waveform, event, physiological and stationarity information. These measures provide information to the clinician about the underlying waveform and event time series, and about the quality of the measurements; providing confidence about the interpretation. Measurements from low quality windows (e.g., low quality because of the waveform, event, stationarity or variability) may be chosen not to be displayed (either graphically or numerically). This assures clinicians that displayed information is at least of intermediate quality.

The quality index 34 is implemented optimally combining the quality measures and the stationarity information using a machine learning model (e.g. using decision trees). The quality index 34 is used to summarize the information from the quality measures into a simple metric which can be used by those clinicians uninterested in the finer details of the quality analysis. The quality report 30, derived from the quality assessment is linked, through a time stamp to the waveform, event and variability information and displayed on the display 14. In addition to the quality report 30, the quality of individual variability calculations 38 can also be displayed as shown in FIG. 3. If one variability measurement is selected on the display 14, the quality report 30 shown for that window can be used to call the waveform and event time series for that window. Similarly, selection of individual measures of variability can cause individual quality measures to be displayed. The physiological filtering 22 and event filtering 24 stages are used to annotate each event in that time series as one of the three categories mentioned above, allowing the clinicians to inspect the waveform and event annotations. The number of quality levels and threshold values on the quality measure to create is modular and can be changed for specific applications. For example, different stationarity requirements could be enforced for certain input types of event time series.

It can be appreciated that the framework described herein may be applied to any physiological waveforms including sets of multi organ waveforms such as the ECG and capnography waveforms which are produced by different organ systems yet are intrinsically related as measure by the cardiopulmonary synchrony (P. Z. Zhang, W. N. Tapp, S. S. Reisman and B. H. Natelson, “Respiration response curve analysis of heart rate variability,” IEEE Transactions on Biomedical Engineering, vol. 44, pp. 321, April 1997). Amongst the two signals, only the ECG has a clearly defined physiological model and morphology and has been extensively studied (Electrophysiology, Task Force of the European Society of Cardiology the North American Society of Pacing, “Heart Rate Variability Standards of Measurement, Physiological Interpretation, and Clinical Use,” Circulation, vol. 93, pp. 1043-1065, March 1996), and (S. Cerutti, A. L. Goldberger and Y. Yamamoto, “Recent Advances in Heart Rate Variability Signal Processing and Interpretation,” IEEE Transactions on Biomedical Engineering, vol. 53, pp. 1, January 2006).

The capnogram has benefited from extensive documentation of tracings (B. Smalhout and Z. Kalenda, An Atlas of Capnography., 2nd ed. The Netherlands: Kerckebosche Zeist, 1981). Prior to the widespread of powerful computers, analysis and measurements were done by hand (measuring angles, visual inspection of shape, and selection of individual breaths for classifiers and detectors), see (B. Smalhout and Z. Kalenda, An Atlas of Capnography., 2nd ed. The Netherlands: Kerckebosche Zeist, 1981), and see (J. M. Goldman and B. H. Dietrich, “Neural network analysis of physiologic waveforms,” in Proceedings of the Annual International Conference of the IEEE Engineering in Medicine and Biology Society, Vol. 13, 1991, pp. 1660).

Limitations of this method may include reproducibility, a reliance on experts with limited availability, and a limit to the number of analyses which may be conducted. To overcome this, the system described herein extends the knowledge gained from HRV and address the limitations in traditional capnograph processing to provide a complete quality assessment for generic physiological waveform inputs. The quality of the signal is ascertained at multiple levels of processing (waveform, events, stationarity), which are specific to variability analysis. The quality process applied to the end tidal CO₂ signal as an example of use in section III, and an example of quality report on the ECG is presented in section IV.

### Obtaining Variability Measures

As discussed above, the quality module 10 may be considered an extension of individual variability measures and analyses to provide quality data 18 with variability waveforms. As such, the generation of a quality report 30 (and other quality data 18) can be applied in any context in which variability measures, obtained from a variability analysis component 12′, can be applied. For example, quality measures can be generated using data obtained in real-time, older data, data obtained in an intensive care unit (ICU), data obtained using portable monitoring devices recording variability, etc. For example, data can be summarized in a mathematical model, which is then used for the computation of quality. A quality assessment of variability therefore is not dependent on any particular mechanism for obtaining the variability data, so long as a set of variability measures is available, and a quality measure can be obtained, as explained in greater detail below. The following illustrates three exemplary monitoring sites 111 (e.g., 111a, 111b, 111c) to demonstrate the various ways in which the variability measures can be obtained in order to generate a quality assessment. Further detail concerning an underlying software framework for obtaining and distributing variability data can be found in applicant's co-pending U.S. patent application Ser. No. 12/752,902, published under US 2010/0261977 and issued as U.S. Pat. No. 8,473,306 to Seely, the entire contents of which are incorporated herein by reference.

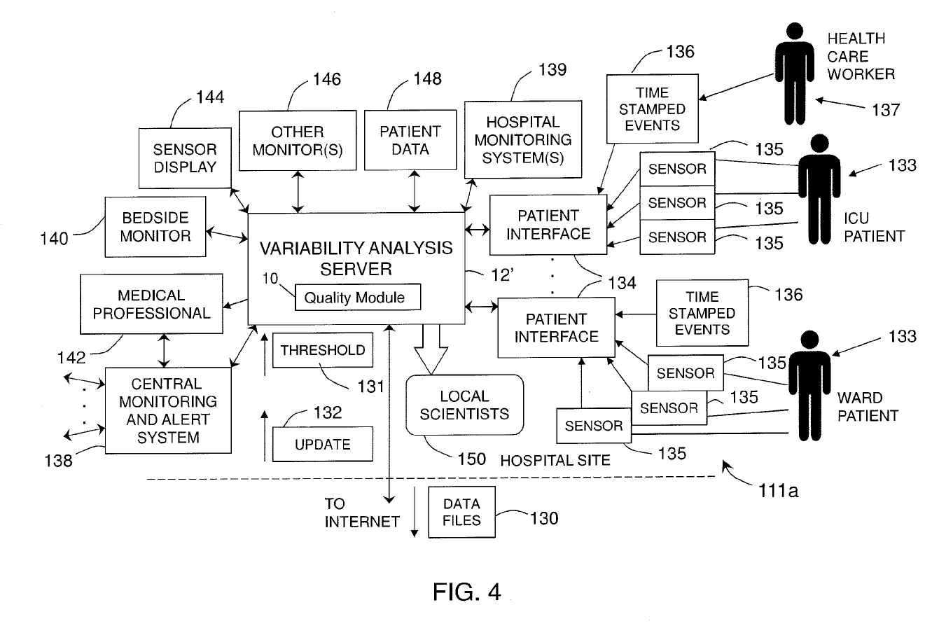

An example of a hospital monitoring site 111a is shown in FIG. 4. The elements shown in FIG. 4 are meant to illustrate several possible components that may interact with one another at the hospital site 111a, however, any number (or all) of these elements can be used or not used in specific hospital sites 111a depending on the actual equipment and/or personnel present at the hospital site 111a and the needs of the patients 133 and personnel. In addition, the parameters being monitored (and the monitors themselves) may differ from network to network. As will be explained, at each monitoring site 111, including the hospital site 111a shown in FIG. 4, is at least one variability analysis server 12′ for using acquired data to conduct variability analyses over time and generate data files 130 that can be viewed at the site and provided to, for example, a central service (not shown). The variability analysis server 12′ includes or otherwise has access to a quality module 10. As shown, each variability analysis server 12′ can interface with multiple patients 133 and, as such, typically only one variability analysis server 12′ is required at each monitoring site 111. The variability analysis server 12′ gathers data acquired from one or more patients 133 through individual patient interfaces 134, computes the measures of variability (i.e. conducts variability analyses) for one or more patient parameters, and connects to, for example, a central server through the Internet, for facilitating the transfer and/or receipt of data files 130, threshold data 131 and update data 132. As shown, there can be different types of patients 133 such as those in the ICU or in a regular hospital ward.

The patient interfaces 134 monitor physiological parameters of the patient 133 using one or more sensors 135. The data or patient parameters can include any variable that can be accurately measured in real time or intermittently. The data may be obtained from a continuous waveform (at a certain frequency level, e.g. 100 Hz for a CO2 capnograph or 500 Hz for an EKG), or taken as absolute measurements at certain intervals, e.g. temperature measurements. The sensors 135 and patient interfaces 134 may include, for example, an electrocardiogram (ECG), a CO₂ capnograph, a temperature sensor, a proportional assist ventilator, an optoelectronic plethymography, a urometer, a pulmonary arterial catheter, an arterial line, an O₂ saturation device and others. To provide more meaning to the data acquired through the sensors 135, clinical events are associated with the data, through an act of recording time stamped events 136, which are typically entered by a heath care worker 137 in the hospital (bedside) environment. Clinical (time stamped) events can be physical activity, administration of medication, diagnoses, life support, washing, rolling over, blood aspiration etc. The clinical events are associated with a specific time, which is then also associated with the data that is acquired at the same specific time using the sensors 315. It will be appreciated that the clinical events can also be recorded in an automated fashion, e.g. by utilizing algorithms which detect events electronically and process such events to designate them as clinical events or noise. In this example, the patient interface 134 is configured to gather the time stamped event data 136 concurrently with the sensor data 135, further detail being provided below. It may be noted that additional non-time-stamped information (e.g. demographics) can also be recorded for each patient.

As can be seen in FIG. 4, the variability analysis server 12′ not only connects to the patient interfaces 134 and the Internet, but also to several other components/entities within the hospital site 111a. For example, the server 12′ can interface with a hospital monitoring system 139 such as a nurse's station, as well as a central monitoring and alert system 138. The central monitoring and alert system 138 is capable of monitoring the variability analyses performed by the variability analysis server 12′ in order to detect critical or potentially critical situations evident from such variability analyses and provide an alert or alerts to a medical professional 142, who can also receive data directly from the variability analysis server 12′. The variability analysis server 12′ can be embodied as a fixed unit or a moveable unit such as on a cart, in order to facilitate movement about the hospital site 111a to serve multiple patients 133 in multiple locations. Similarly, the variability analysis server 12′ can be a proprietary apparatus or can be embodied as an add-on to existing beside or centralized equipment to minimize space.

The variability analysis server 12′ can also interact with a bedside monitor 140, which may be made available to or otherwise represent a nurse or other personnel that monitors the patient 133 at the bedside. Similarly, the variability analysis server 12′ can also interact with sensor displays 144, which are associated with other medical equipment such as ECGs, blood pressure sensors, temperature sensors etc. As noted above, the variability analysis server 12′ can be a separate, stand-alone unit but may also be integrated as a plug-in or additional module that in this case could be used or integrated with existing bedside monitoring equipment, displays and sensors. FIG. 4 also shows other monitors 146 which can include any other monitoring system or equipment that either can provide useful medical data or patient data 148 or would benefit from the data acquired by the variability analysis server 12′. Patient data 148, e.g. provided by an electronic patient database (not shown) or manually entered can also interact with the variability analysis server 12′. As will be discussed below, the patient data 148 may be appended to, or included with the data files 130 to provide further context for the data contained therein. This enables patient specifics such as age, general health, sex etc. be linked to the acquired data to assist in organizing data into demographics. As also shown in FIG. 4, the variability analysis server 12′ can provide data or otherwise useful information for local scientists 150 that are interested in or involved in the implications and effects of variability. It will be appreciated that patient privacy and other concerns can be addressed as required, by adding data security or other de-identification measures.

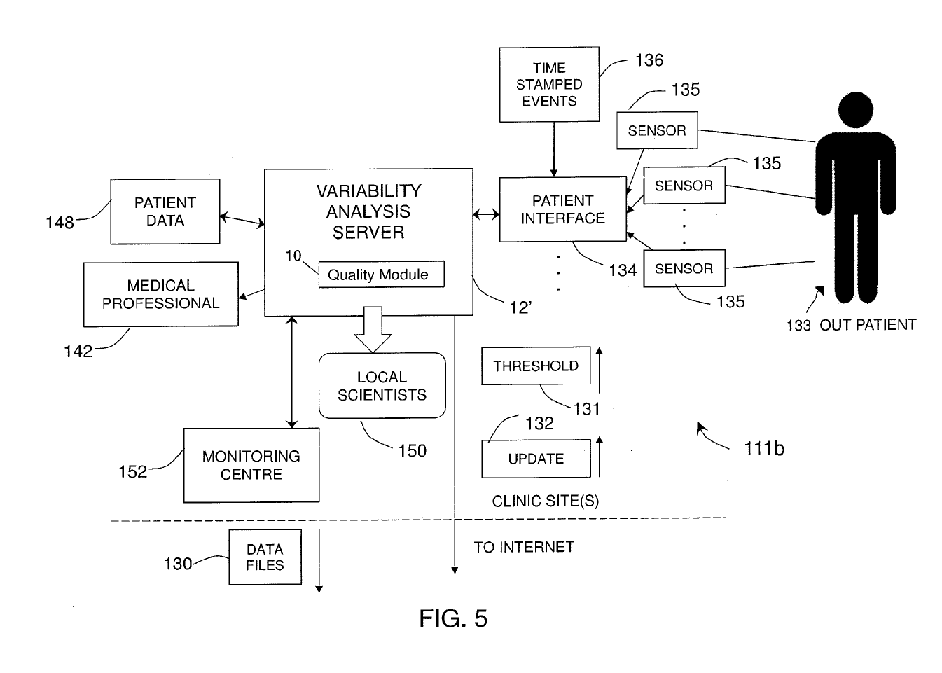

Turning now to FIG. 5, a clinic site 111b is shown. An example of a clinic site 111b is a bone marrow transplant clinic. Similar to the hospital site 111a discussed above, the clinic site 111b includes a variability analysis server 12′, that obtains data from one or more patient interfaces 134, and connects to the Internet for facilitating data transfer (i.e. to send data files 130 and to receive threshold data 131 and update data 132). In the clinic site 111b, the patients 133 are referred to as outpatients as they are not admitted to a hospital. The sensors 135, clinical events recorded as time stamped events 136 and patient data 148 is acquired and used in a manner similar to that discussed above and thus further details need not be reiterated. Similarly, the variability analysis server 12′ can provide data and interact with medical professionals 142 at the clinic site 111b, as well as local scientists 150, if applicable. The clinic site 111b may include one or more variability analysis servers 12′, and would typically include a monitoring center 152 that monitors the analyses of the various outpatients 133 and provides alerts if necessary. The monitoring center 152 enables the clinic's variability analysis server 12′ to be monitored from a remote location and allows personnel to monitor several servers 12′ if several are present in the clinic. In this way, a central monitoring center 152 can be used to service several clinic sites 111b.

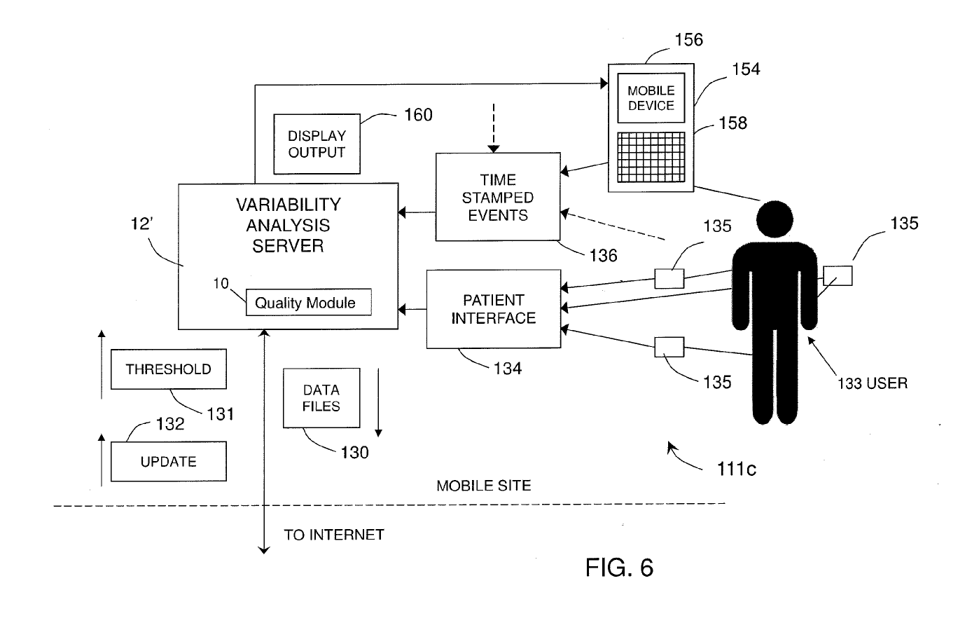

A mobile site 111c is shown in FIG. 6. The mobile site 111c enables the capabilities of the variability analysis server 12′ to be used outside of the hospital and clinical environments and, as such, in this embodiment, the mobile site 111c serves any “user” or “subject”. For the sake of consistency, hereinafter the term “patient” will refer collectively to any user or subject. In this way, it may be appreciated that variability analyses can be performed on any user, including athletes, firefighters, police officers, or any other person that can benefit from monitoring variability of one or more physiological parameters. This can therefore extend to providing real-time monitoring in extreme environments such as during a fire, in a mine, during rescue missions etc. where variability can indicate a potentially critical situation. In all cases, variability can be monitored over time and analyzed on an individual basis for any patient 133 such that the resultant data is specific to that individual. Using the wider system allows a central service to take advantage of the individual results for many patients 133 and ascertain further and more complete information. The mobile site 111c generally represents any site that includes a variability analysis server 12′, which connects to the system 8 and can communicate with one or more patients 133, whether they are patients in the traditional sense or another type of user.

In the example shown in FIG. 6, the user 133 generally includes a mobile device 154 and has a number of sensors 135 that are in communication with a variability analysis server 12′. The mobile device 154 can also be used to provide inputs, e.g. for the time stamped event data 136, as well as to provide a display to the user 133 for entering parameters or to view display data 160 acquired by the sensors 135 and/or processed by the server 12′. The connections between the mobile device 154 and the server 12′, as well as between the sensors 135 and patient interface 134 can be wired or wireless and the variability analysis server 12′ can be a fixed unit at a base station or a portable unit such as on a cart at a monitoring center. The mobile device 154 can be a personal digital assistant (PDA), mobile telephone, laptop computer, tablet computer, personal computer, or any other device that can provide an input device, a display and some form of connectivity for interacting with the variability analysis server 12′, preferably in a completely mobile manner.

As noted above, each monitoring site 111 may include a variability analysis server 12′. Details of various embodiments of existing variability analysis apparatus and configurations can be found in U.S. Reissue Pat. No. RE41,236 E to Seely, the entire contents of which are incorporated herein by reference.

### Section II

### Examples and Embodiments

An example of a quality assessment for the purpose of variability monitoring is now provided.

### A—Method to Assess Waveform Quality for Variability Analysis:

i) The assessment diagrammed in FIG. 3 may be applied to any physiological waveforms including sets of multi organ waveforms 32 such as the ECG and capnography waveforms 32. The waveforms 32 may be acquired as stored waveforms 32 in a database or directly from patient monitors as raw sensor data or processed waveforms. The transformation of a recorded physiological signal into a high quality event time series begins at the waveform level. The waveform quality stage 20 shown in FIG. 3 comprises a threefold process to ascertain the useable portions of the waveform 32.

ii) The analysis of the waveform quality 20 can comprise an analysis for a) disconnections; data segments identified as being outside monitor range (i.e. negative value on a breathing rate monitor), b) saturations in the signal and gross amplitude changes and c) wandering baselines.

### B—A Method of Physiological Filtering for Variability Analysis:

i) The modular quality framework described herein accepts at this point data provided as a time series of events (if waveform 32 is not available due, for example, to data storage constraints). However quality analyses relying on the waveform interpretation may not be feasible in such a situation.

ii) Waveform portions not identified as disconnections represent useable portions of the signal for the purpose of segmentation. Unusable portions create gaps, or interruptions in the signal. Waveforms 32 are segmented into events using methods appropriate to the input waveform 32. For example R-peak detectors may be appropriate for ECG signals, and zero-crossing detections may be appropriate for breathing signals centered on zero.

iii) Following the segmentation into events, the method comprises comparing event measurements to literature standards to determine at stage 22, if each event meets appropriate physiological limits. The criteria can include boundaries on event duration and event amplitude.

### C—A Method of Event Filtering for Variability Analysis:

i) Further stratification of events creating three event categories: 1) non-physiological, 2) physiological, and 3) physiological and high quality.

ii) The event filtering stage 24 of the assessment is variable in scope and modular and comprises any of the three following methods. Some methods of event quality discrimination may be accomplished at this stage comprise:

a) Event quality discrimination through segmentation involving a comparison with a template or population norms,

b) Event quality discrimination involving multichannel signal comparison (e.g. see Q. Li, R. G. Mark and G. D. Clifford, “Robust heart rate estimation from multiple asynchronous noisy sources using signal quality indices and a Kalman filter,” Physiol. Meas., vol. 29, pp. 15-32, January, 2008 and B. Krauss, A. Deykin, A. Lam, J. J. Ryoo, D. R. Hampton, P. W. Schmitt and J. L. Falk, “Capnogram Shape in Obstructive Lung Disease,”Anesthesia & Analgesia, vol. 100, pp. 884, March, 2005), and

c) Event quality discrimination comprising of ectopic filtering which can involve statistical rules or outlier detection algorithms (e.g. see Q. Li, R. G. Mark and G. D. Clifford, “Robust heart rate estimation from multiple asynchronous noisy sources using signal quality indices and a Kalman filter,” Physiol. Meas., vol. 29, pp. 15-32, January 2008; and S. Nemati, A. Malhotra and G. Clifford, “Data Fusion for Improved Respiration Rate Estimation,” EURASIP} Journal on Advances in Signal Processing, vol. 2010, pp. 926305, 2010).

### D—A Method to Accomplish Variability Calculation:

i) Variability analysis at stage 36 comprising variability measures chosen from a plethora of over 100 measures from four domains, and calculations are performed on the high quality event time series.

ii) The present modular framework can allow selectable variability measures for organ specific or signal specific quality assessment report for variability analyses.

iii) These calculations, representing the complexity of an event time series, are performed over a set window length.

iv) A variability analysis comprising of multiple windows to report the changes in variability over time.

v) An event-based variability analysis applied to portions of signal before and after an event, and selected for a window based analysis.

### E—A Method for Stationarity Assessment for Variability Analysis:

i) The windowed high quality event time series signal is subject to a stationarity assessment stage 28 which comprises the use of models applied to windows of high quality event time series. Data models to assess the stationarity of the high quality event time series in a window may include linear trend, spike and step models.

ii) The models in the stationary assessment stage 28 may make use of customizable thresholds. One method to select thresholds is by analysis of the histogram and cumulative distribution function of the stationarity models applied to a dataset. For example, thresholds may be selected to retain 95% of data points.

iii) In cases of non-stationary windows, the quality index, Q, may be automatically downgraded to zero or “Low” quality, which removes or alters the display of the variability for that window, to ensure inappropriate measurements are interpreted appropriately.

### F—A Method to Implement Quality Measures Q & Qv:

i) Quality measures 26, Q, are derived from the waveform quality stage 20, physiological filtering stage 22, event filtering stage 24, and stationarity assessment stage 28 to represent the quality of the waveform and events within a window as a single number.

ii) Certain variability measures are more sensitive to the quality of the input waveform, therefore to the quality of the variability measurements is also assessed using intrinsic and extrinsic parameters to produce a confidence interval about the variability measurements. Intrinsic parameters may be goodness of fit values embedded in the algorithms themselves while extrinsic factors may include statistics from the waveform and event quality processing such as percentage of removed data.

iii) Quality indices 34 are derived from quality measures 26 and stationary assessment stage 28 to represent the quality of the variability measures for a window, as a single number.

iv) Quality indices can comprise of measure of the signal that are within the range [0,1] such as:
a) Percentage time spent in physiological ranges
b) Percentage time spent in high quality intervals
c) Longest uninterrupted series of normal events
d) Percentage time reporting a disconnection
e) Percentage time reporting saturation.
f) Degree of stationarity

v) Quality indices 34 can comprise a measure of the signal that are not necessarily in the range [0,1] such as:
a) Number of gaps (a gap being a discontinuity) in window due to waveform quality analysis (e.g., saturation and disconnection).
b) Number of gaps in window due to physiological filtering
c) Nature of gaps in window due to physiological filtering

vi) The window-based quality indices may be combined using equal or unequal weights to produce a value between [0,1]. For example, the use of weights, {A,B,C,D,E,F}, where the sum of the weights is 1 can be combined with quality indices 34 {a,b,c,d,e,f} in the following manner:

Q=a*A+b*B+c*C+d*D+e*E+f*F

vii) Qv may be obtained in the same manner as Q, using quality indices relevant to the variability measures (i.e. intrinsic and extrinsic quality indices 34).

viii) Qv may comprise of an array of values, representing one Qv for each individual quality measure.

ix) The indices Q and Qv represents a summative value of the quality for a window.

### G—A Method to Implement Quality Index, QI:

i) The transformation of Q to a quality index 34 QI comprises thresholding or the use of machine learning models to create distinct quality levels. The quality index 34 can comprise any number of categories, for example “Low”, “Moderate” and “High”.

ii) The QI 34 provides an ‘at a glance’ summary of the quality of the waveform, event time series and stationary of a window.

iii) The quality system for variability analysis comprises the integration of the waveform, events and quality index 34 with the variability measurements for viewing waveform and for the continuous monitoring of variability.

### H—A Method to Display Quality of Waveform, Event and Variability for Variability Analysis:

i) QI and Qv form a per-window quality report 30 which may be displayed by clicking on a point in the variability graph display.

ii) The quality index QI 34 and Qv 26 are displayed (either numerically, or as a waveform), and used to transform the displayed variability. Instances of “Low” quality for a window or time period, removes the display of the variability for that window, to ensure inappropriate measurements are interpreted appropriately. When Qv is applied to individual variability measures, low values of (numerically or as a waveform), Qv remove the display for the offending windows, so as to not display measures for which the interpretation could be problematic.

iii) The quality report 30 (quality indices and quality index) per window serve as alerts that link to using a time stamp, to the waveform, the event time series, and the variability.

iv) The quality report 30 can be used to flag problems in data and has other uses, such as linking to the waveform where the events are delineated and their quality (non-physiological, physiological or physiological and high quality is indicated).

v) Providing quality of results provides benefits over viewing the variability to aid in interpretation of analysis and indicator of data quality in raw, event, high quality event and window.

vi) The variability is displayed at the same time as the waveform and event time series, either numerically or graphically.

vii) Variability results displayed numerically and graphically, and low quality for QI or Qv removes display for those widows. This assures clinicians that displayed information is at least of intermediate quality.

viii) The quality report, derived from the quality assessment is linked, through a time stamp, to the waveform, event and variability information. If one variability measurement is selected on a display, the quality report shown for that window can be used to call the waveform and event time series for that window.

ix) The physiological and event filtering stages are used to annotate each event in that time series as one of the three categories mentioned above, allowing the clinicians to inspect the waveform and event annotations.

x) The number of quality levels and threshold values on the quality measure to create is modular and can be changed for specific applications. For example, different stationarity requirements could be enforced for certain input waveforms types.

xi) Input feedback 19 can be provided by the user in order to enhance the quality assessment.

### Section III

### A Quality Analysis Applied to Capnography

### A—Instrumentation and Measurements

As an example, the quality framework can be applied to capnography data. A monitor that can collect the capnography signal is the Philips Intellivue which records the end tidal CO2 signal in millimeters of mercury (mmHg) at a sampling frequency of 125 Hz.

### B—Waveform Quality Stage 20

The waveform quality is measured by an analysis for disconnections and saturation events of duration ≧1 s. The sampling rate of the signal can be an input to the system or derived from the signal timestamps. Disconnections may be found by looking for consecutive values outside the monitor range. These ranges may be monitor specific, and are generally outside normal physiological values.

### C—Physiological Filtering Stage 22

The waveform is segmented into events with a fixed threshold at 10 mmHg. Crossings preceded by a smaller value than the threshold level are considered to be indicative of expiration, otherwise they are inspirations. The distance between two consecutive events is the interbreath interval. The expiration or inspiration time may be used to determine the interval, as can any other signal feature such as time of peak pressure.

Each event's duration and maximal amplitude are measured. Events were classified as physiological or non-physiological using duration and amplitude measurements (between 1 and 15 seconds and equal or above 20 mmHg at the highest point of the event) and non-physiological events (outside duration range or below 20 mmHg at the highest point of the event).

### D—Event Filtering Stage 24

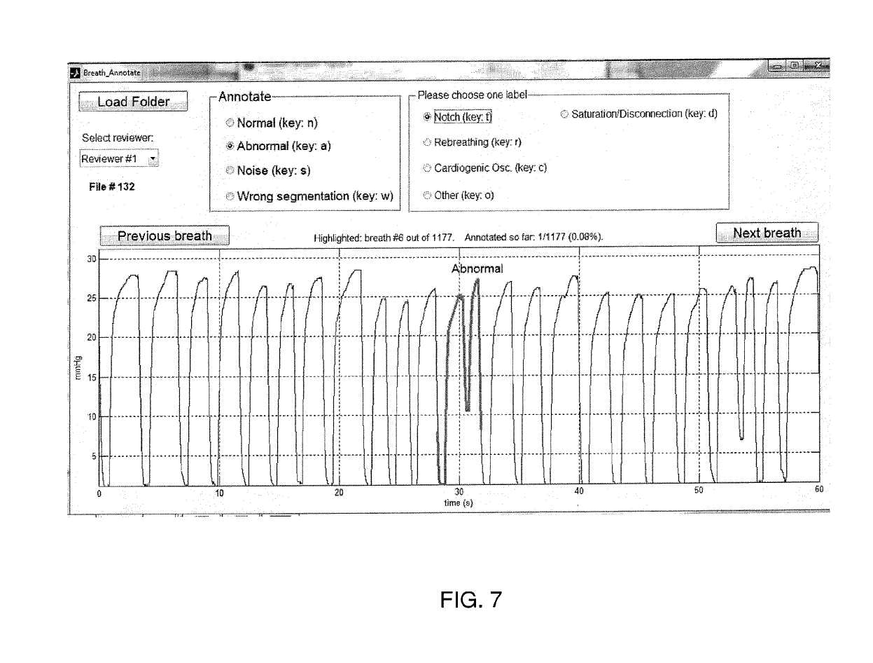

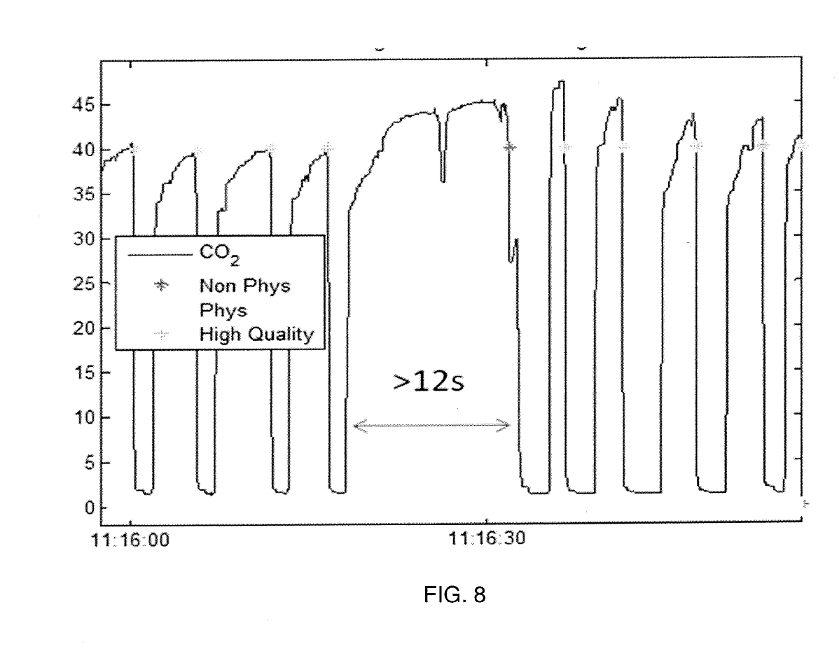

In the absence of a gold standard for the identification of high quality breaths, an expert analysis was used to determine normal versus abnormal events. Six experts annotated over 10 000 individual breaths. For each of these breaths, 15 parameters were measured and used as input for a single class classifier which is then applicable to new capnogram signals in a reproducible way. The events classified as normal by the classifier are labeled ‘high quality’, because in addition to meeting physiological requirements, their morphology is classified as normal. Confidence about the quality of the event is increased. FIG. 7 shows the software that was built to allow experts to annotate breaths, and FIG. 8 shows an example of a minute long capnogram with one breath identified as non-physiological.

### E—Variability Calculation Stage 36

Variability metrics appropriate to the calculation of reparatory parameter variability were selected. Variability may be calculated on the event time series of event duration, or other parameters measures from the waveform such as are under the curve. Parameters for the variability metrics were selected appropriately for this time series, as was the window length over which the variability were calculated.

### F—Stationarity Assessment Stage 28

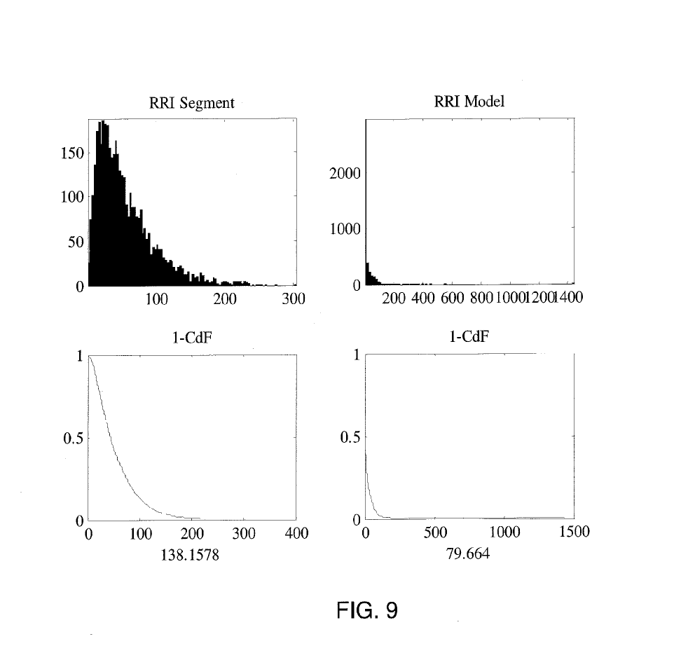

The linear trend, spike and step model were applied to 2000 windows of high quality event time series which produced the histograms seen in FIG. 9. From the cumulative distribution function, thresholds were selected to retain values within a 95% range of the values for which stationarity was calculated. The degree of stationarity is used as a quality measure 26 in the quality report 30 per window. It can also be used in the equation for the quality of the variability measures. The degree of non-stationarity also relates to the quality of individual variability measures, as measure can vary in robustness with respect to the stationarity of the event time series. This result can be used in the quality report 30.

### G—Quality Measure 26 and Quality Index 34

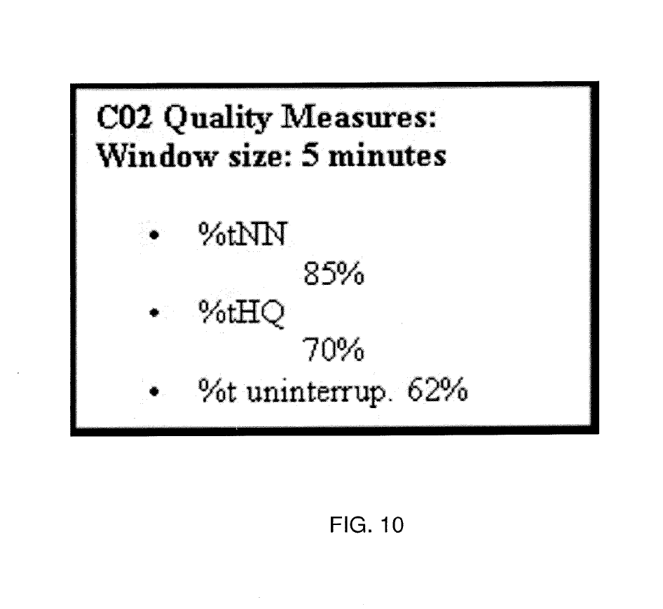

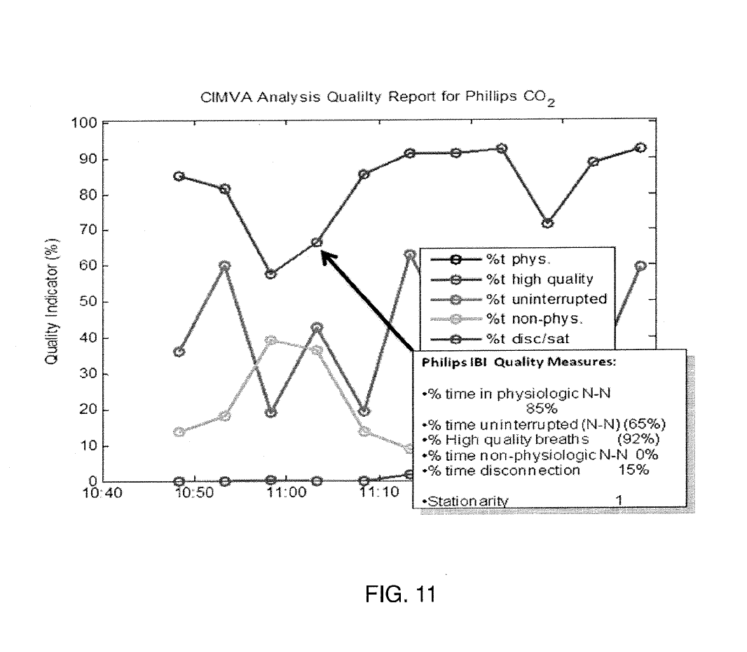

Three quality measures 26 were used to form a composite quality measure. They are 1) percentage time in normal-to normal event intervals (% tNN), 2) percentage time high quality (% tHQ) and 3) percentage time uninterrupted (% t uniterrup) were used to create the quality index. Other measures such as percentage time in non-physiological intervals (% t non-phys.) and percentage time of disconnection or saturation in the waveform (% t disc/sat) are calculated, reported in the quality report but not used in the quality index calculation. FIG. 10 presents a quality report for a particular window. FIG. 11 presents an example of a quality report showing the quality measures, which are calculated every window.

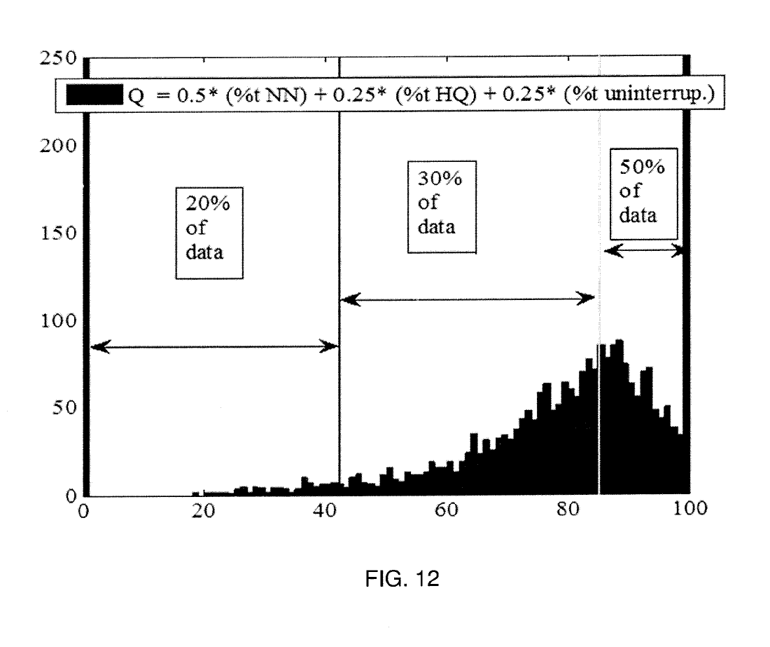

FIG. 12 demonstrates the histogram of the composite quality measure of 2000 windows. The composite was calculated using three quality measures using weights of 0.5, 0.25 and 0.25. The legend of the figure shows the names of the quality measures as well as the weighing used for the composite measure. The histogram was used to determine thresholds to represent generic quality levels which impact the displayed variability. The vertical lines represent threshold levels indicating low, moderate and high quality for the quality index.

### H—Displayed Variability 16

FIG. 12 also indicates that a composite quality measure 26 between 0 and 42 will be converted to Low quality, and the variability can be configured to not be displayed for these windows. This allows the clinician to have confidence in the displayed variability measurements because windows with low quality are not displayed. The windows with a composite quality measure between 42 and 85 are considered moderate quality. The variability measurements for these windows are displayed in a shaded colour. Windows with a composite quality measure above 85 are considered high quality. This method allows the clinical to have confidence in the displayed variability waveform and greatly reduces the possibility that the clinician will interpret variability measurements which were performed on data of low quality. More stringent, or more liberal thresholds could be chosen for example, to accept windows from noisy waveforms.

### Section IV

### Quality Report for ECG Waveform

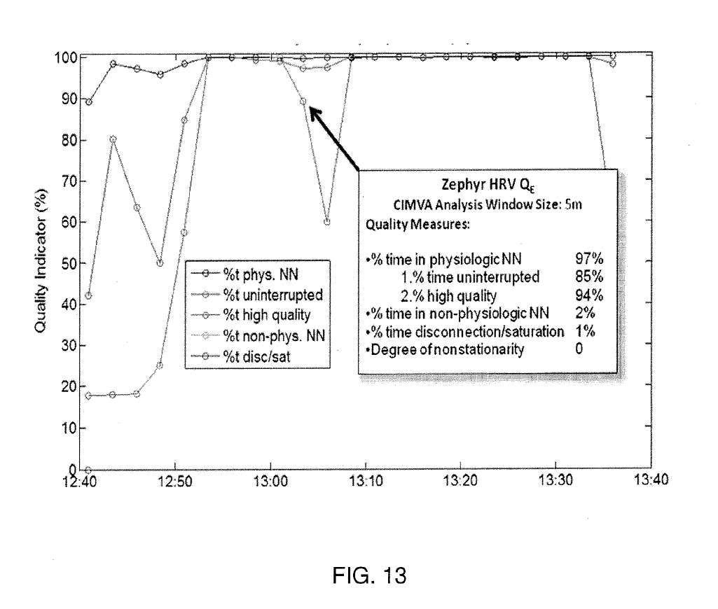

FIG. 13 is an example of the implementation of the method to the ECG. In the case of HRV, published quality algorithms such as the SQI (see Q. Li, R. G. Mark and G. D. Clifford, “Robust heart rate estimation from multiple asynchronous noisy sources using signal quality indices and a Kalman filter,” Physiol. Meas., vol. 29, pp. 15-32, January 2008) can be used as an input the composite quality measure.

### Section V

### Conclusions

It is highly desirable to provide a quality assessment of the waveform and quality measures 26 to provide a confidence interval about the resulting measures (output variability matrix), along with the variability measures to ensure the correct interpretation of the measure. The presented framework assesses quality at the waveform, event, and variability measurement level, in addition to assessing stationarity. Together, these components form a complete quality assessment for the purpose of variability analysis.

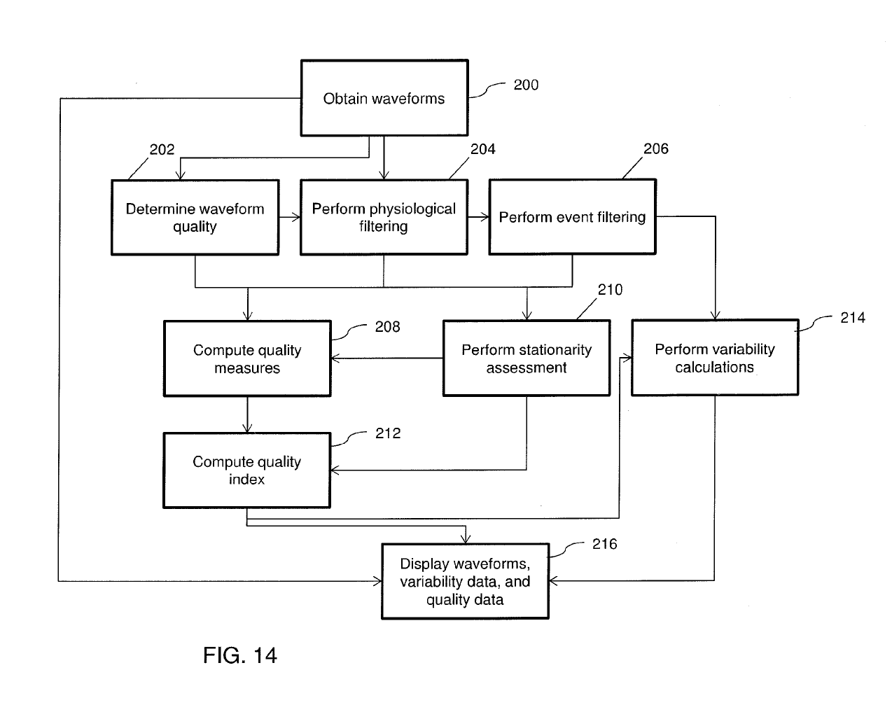

FIG. 14 illustrates a flow chart illustrating an example of a set of computer executable instructions that may be executed in performing a quality assessment on variability data. At 200 the system obtains the waveforms, e.g., as acquired from multiple sensors interfacing with multiple organs. The waveform quality is determined at 202, physiological filtering is performs at 204, and event filtering is performed at 206. It can be appreciated from FIG. 14 that the various calculations can feed into one another, in order to enable a quality measure to be computed at 208. For example, the operations performed at 202, 204, and 206 may feed into both the computation at 208 and a stationarity assessment performed at 210. Similarly, the results of the event filtering at 206 may feed into the quality measurement at 208, the stationarity assessment at 210, and variability calculations performed at 214 (i.e. to have variability calculations performed on cleaned event time series as shown in FIG. 3).

The quality index may then be computed at 212 using the quality measures and the results of the stationarity assessment 210 as explained above. The quality index 212, the waveforms themselves, and the variability data output from the variability calculations may then be displayed for the user at 216.

It will be appreciated that any module or component exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the system, any component of or related to the system, etc., or accessible or connectable thereto. Any application or module herein described may be implemented using computer readable/executable instructions that may be stored or otherwise held by such computer readable media.

The steps or operations in the flow charts and diagrams described herein are just for example. There may be many variations to these steps or operations without departing from the principles discussed above. For instance, the steps may be performed in a differing order, or steps may be added, deleted, or modified.

Although the above principles have been described with reference to certain specific examples, various modifications thereof will be apparent to those skilled in the art as outlined in the appended claims.

1. A method of assessing quality for a variability analysis, the method comprising:
obtaining at least one waveform corresponding to a corresponding physiological measurement;
determining at least one measure of waveform quality of the at least one waveform;
extracting from a waveform, at least one event time series;
determining a measure of event time series quality of the at least one event time series;
determining at least one measure of stationarity of the at least one event time series;
computing a quality measure using the at least one measure of waveform quality and the at least one measure of stationarity; and
displaying the quality measure.

2. The method of claim 1, wherein the quality measure is displayed with variability data.

3. The method of claim 2, further comprising performing a variability analysis on the at least one event time series, and displaying the variability data.

4. The method of claim 1, further comprising computing a quality index using at least one quality measure.

5. The method of claim 4, wherein the quality index is computed using a threshold applied to a range for the quality measure.

6. The method of claim 1, wherein determining the at least one measure of waveform quality further comprises performing physiological filtering to remove at least one event from the at least one event time series.

7. The method of claim 1, wherein determining the at least one measure of waveform quality further comprises removing at least one segment of a waveform signal.

8. The method of claim 1, wherein determining the at least one measure of stationarity comprises removing at least one segment of the time series.

9. The method of claim 1, wherein determining the at least one measure of waveform quality measure further comprises performing event filtering on events detected from the at least one waveform.

10. The method of claim 1, wherein determining the waveform quality comprises analyzing the at least one waveform for at least one of disconnections, saturations in the signal, and wandering baseline.

11. The method of claim 1, wherein the at least one waveform is displayed with the quality measure.

12. The method of claim 1, further comprising at least one of:
performing a variability analysis on the at least one event time series at least one waveform, and displaying variability data, wherein the quality measure is displayed with variability data;
computing a quality index using the at least one measure of waveform quality, wherein the quality index is computed using at least one of a threshold applied to a range for the quality measure and a mathematical model;
wherein determining the at least one measure of waveform quality further comprises performing physiological filtering to remove at least one event from the at least one event time series;
wherein determining the at least one measure of waveform quality further comprises removing at least one segment of a waveform signal;
wherein determining the at least one measure of stationarity comprises removing at least one segment of the time series;
wherein determining the quality measure further comprises performing event filtering on events detected from the at least one waveform;
wherein determining the waveform quality comprises analyzing the at least one waveform for at least one of disconnections, saturations in the signal, and wandering baselines; and
wherein the at least one waveform is displayed with the quality measure.

13. A computer readable storage medium comprising computer executable instructions for performing the method of claim 1.

14. A system comprising a processor and memory, the memory comprising computer executable instructions for performing the method of claim 1.
